(19) United States
(12) Patent Application Publication
(10) Pub. No.: US 2013/0116762 A1
(43) Pub. Date: May 9, 2013

(54) ORAL SURGERY RECOVERY MASK

(71) Applicant: Ernest Lai, Honolulu, HI (US)

(72) Inventor: Ernest Lai, Honolulu, HI (US)

(21) Appl. No.: 13/425,731

(22) Filed: Mar. 21, 2012

Related U.S. Application Data
(60) Provisional application No. 61/557,657, filed on Nov. 9, 2011.

(51) Int. Cl. A61F 7/10

(52) U.S. Cl. 607/109

(57) ABSTRACT

One example embodiment includes an oral surgery recovery mask for cooling an area of a user's face. The oral surgery recovery mask includes a bladder receptacle. The bladder receptacle includes a first layer and a second layer at least partially secured to the first layer. The oral surgery recovery mask also includes a cooling liquid, where the cooling fluid is configured to be inserted into the bladder receptacle. The oral surgery recovery mask also includes a securing device, where the securing device is configured to hold the oral surgery recovery mask in place relative to the user.

### CROSS-REFERENCE TO RELATED APPLICATIONS

This application claims the benefit of and priority to U.S. Provisional Patent Application Ser. No. 61/557,657 filed on Nov. 9, 2011, which application is incorporated herein by reference in its entirety.

### BACKGROUND OF THE INVENTION

The health benefits of cooling a particular body part have long been known. Cooling helps to reduce inflammation and helps to dull pain sensations. The use of cooling is particularly important following injury or after medical and dental procedures involving surgery. This is when the cooling can have the most beneficial effects. In some instances, if cooling is not done immediately, then the cooling is totally ineffective. In surgical procedures performed by Oral Maxillofacial Surgeons and Dentists, routine out-patient procedures include the removal of wisdom teeth (3^{rd} molars). The location of these teeth often located in the angle of the mandible near the exterior ear canal. Pain and Inflammation often radiates from this region into more superior areas posterior to the ear and into the temporal area of the head. Other common surgeries include dental implants and sinus lifts, whereby these procedures typically present post-operative pain, inflammation, and swelling which radiate from any number of areas in the maxilla, mandible, cheek, and suborbital regions.

There are some drawbacks to cooling. In particular, it is often difficult to cool only a specific area. For example, cold packs are often placed on the site. However, the area cooled is the same whether the area is an ankle or a finger; the area is equal to the area covered by the cold pack. This means that the area around the injury or surgical site may be cooled as well. This may also mean that the user experiences discomfort in the surrounding area due to the excessive cooling.

In addition, the cooling may not be limited. I.e., the cooling may not be targeted to the desired area or the desired length of time. This is because the cooling lasts as long as the cold pack is cold. How long the cold pack is cold depends, in turn, on a number of factors such as ambient temperature, size of the cold pack and other factors.

Moreover, the cold pack is difficult to conform to the user's anatomy. I.e., the cold pack comes in a particular shape, and retains that shape. It cannot be wrapped or otherwise molded to the user. There are systems which retain the cold pack in particular areas; however, these systems tend to be bulky and unwieldy.

Accordingly, there is a need in the art for an oral surgery recovery mask that can target only a portion of a user's face. In addition there is a need in the art for the oral surgery recovery mask to be compact, so that the mask does not increase the user's discomfort.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes an oral surgery recovery mask for cooling an area of a user's face. The oral surgery recovery mask includes a bladder receptacle. The bladder receptacle includes a first layer and a second layer at least partially secured to the first layer. The oral surgery recovery mask also includes a cooling liquid, where the cooling fluid is configured to be inserted into the bladder receptacle. The oral surgery recovery mask also includes a securing device, where the securing device is configured to hold the oral surgery recovery mask in place relative to the user.

Another example embodiment includes an oral surgery recovery mask for cooling an area of a user's face. The oral surgery recovery mask includes a bladder receptacle. The bladder receptacle includes a first layer, where the first layer includes plastic. The bladder receptacle also includes a second layer where the second layer includes plastic and at least partially secured to the first layer. The oral surgery recovery mask also includes a cooling liquid, where the cooling fluid is configured to be inserted into the bladder receptacle. The oral surgery recovery mask further includes a securing device. The securing device includes a first ear strap and a second ear strap. The securing device also includes micro suction elements, where the micro suction elements are configured to adhere the bladder receptacle to the face of the user. The oral surgery recovery mask additionally includes a wire frame, where the wire frame is configured to allow the user to change at least a portion of the shape of the oral surgery recovery mask.

Another example embodiment includes a method for using an oral surgery recovery mask for cooling an area of a user's face. The method includes refrigerating the oral surgery recovery mask. The method also includes placing the oral surgery recovery mask on a user. The method further includes securing the oral surgery recovery mask on the user.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of an oral surgery recovery mask;

FIG. 2 illustrates a cross-section of an oral surgery recovery mask;

FIG. 3A illustrates a perspective view of the oral surgery recovery mask in use;

FIG. 3B illustrates a bottom perspective view of the oral surgery recovery mask in use;

FIG. 4 illustrates an alternative example of an oral surgery recovery mask;

FIG. 5 illustrates an example of the alternative oral surgery recovery mask in use; and

FIG. 6 is a flow chart illustrating a method of using an oral surgery recovery mask.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

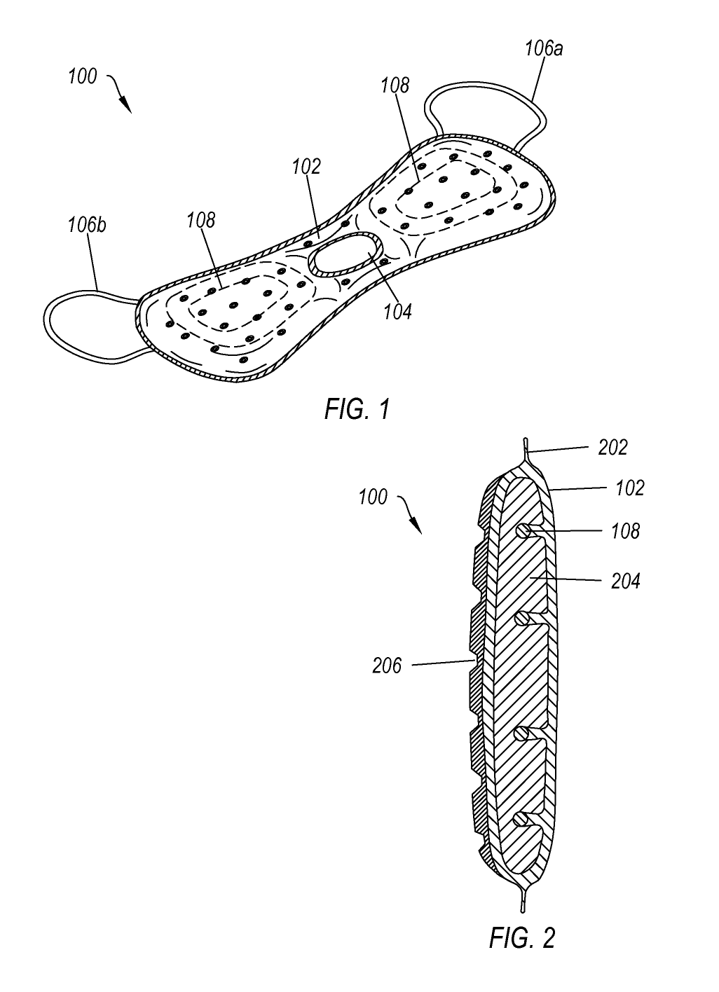

FIG. 1 illustrates an example of an oral surgery recovery mask 100. In at least one implementation, the oral surgery recovery mask 100 can be used to cool portions of a user's face and/or neck. For example, the oral surgery recovery mask 100 can be used to cool a user's jaw after oral surgery. The oral surgery recovery mask 100 can help prevent or reduce inflammation. I.e., the oral surgery recovery mask 100 can cool a specific area that may be prone to inflammation, reducing the likelihood of inflammation and increasing healing times.

FIG. 1 shows that the oral surgery recovery mask 100 can include a bladder receptacle 102. In at least one implementation, the bladder receptacle 102 can include a pouch for retaining fill material. I.e., the bladder receptacle can include any device for accepting and retaining a cooling fluid. In particular, the bladder receptacle 102 can be configured to target the cooling effects of the oral surgery recovery mask 100 to deliver cooling to only a desired area.

In at least one implementation, the bladder receptacle 102 can be made of any desired material. For example, the bladder receptacle 102 can include two sheets of plastic. The sheets of plastic can be filled with the desired cooling fluid then sealed, permanently retaining the cooling fluid. Additionally or alternatively, the bladder receptacle 102 can include one or more valves to allow cooling fluid to be added or removed as desired. Additionally or alternatively, the bladder receptacle 102 can be open to accommodate cooling packs or other devices. I.e., a separate cooling pack can be inserted and removed from the bladder receptacle as desired to provide cooling.

Additionally or alternatively, the bladder receptacle 102 can be any desired shape. For example, the bladder receptacle 102 as shown in FIG. 1 is symmetrical. However, one of skill in the art will appreciate that the bladder receptacle 102 can be shaped to allow the oral surgery recovery mask 100 to cool a specific area on the user's face. E.g., the bladder receptacle 102 can be wider in the center portion, allowing more cooling around the user's mouth or portions may be removed to allow the oral surgery recovery mask 100 to cool only one side of the user's face.

FIG. 1 also shows that the oral surgery recovery mask 100 can include a chin cut out 104. In at least one implementation, the chin cut out 104 can allow proper placement of the oral surgery recovery mask 100. In particular, the chin of the user will at least partially rest in or protrude through the chin cut out 104. This can ensure that the bladder receptacle 102 rests against the desired area and remains in place, as described below.

FIG. 1 further shows that the oral surgery recovery mask 100 can include a first ear strap 106a and a second ear strap 106b (collectively “ear straps 106”). In at least one implementation, the ear straps 106 can ensure proper orientation of the oral surgery recovery mask 100. I.e., the chin cut out 104 can properly orient the center of the oral surgery recovery mask 100 and the ear straps 106 can properly orient the lateral portion so the oral surgery recovery mask 100 is properly placed. Additionally or alternatively, the ear straps 106 can ensure that the oral surgery recovery mask 100 remains in place. I.e., the ear straps 106 can create a tension in the oral surgery recovery mask 100, ensuring that the oral surgery recovery mask 100 remains in place during use.

In at least one implementation, the ear straps 106 can be made of any desired material. For example, the ear straps 106 can be made of an elastic material. E.g., the ear straps 106 can be stretched to accommodate the user's ears and then retract when the tension is removed. This allows the ear straps 106 to be retained in place, which in turn retains the oral surgery recovery mask 100 in the desired position.

FIG. 1 additionally shows that the oral surgery recovery mask 100 can include a wire frame 108. In at least one implementation, the wire frame 108 can allow the user to customize the shape of the oral surgery recovery mask 100. I.e., the wire frame 108 can add rigidity to the oral surgery recovery mask 100, allowing the oral surgery recovery mask 100 to conform to the shape of the user's face. In particular, the more cooling fluid within the bladder receptacle 102, the more flaccid the oral surgery recovery mask 100 becomes. The wire frame 108 can allow additional cooling fluid to be placed within the oral surgery recovery mask 100 without losing rigidity that ensures that the cooling fluid remains against the skin of the user.

FIG. 2 illustrates a cross-section of an oral surgery recovery mask 100. In at least one implementation, the oral surgery recovery mask 100 can be used to cool a desired area of a user's face. In particular, the oral surgery recovery mask can be cooled and then applied to a user's face, cooling the user's face in turn.

FIG. 2 shows that the bladder receptacle 102 can includes a seal 202. In at least one implementation, the seal 202 can include a portion of the bladder receptacle 102 where the sheets are attached to one another. I.e., the seal 202 can include a portion of the bladder receptacle 102 where the first layer of material and the second layer of material are joined together. The seal 202 can be formed using adhesive, pressure, heat or any other desired method.

FIG. 2 also shows that the oral surgery recovery mask 100 can include a cooling fluid 204. In at least one implementation, the cooling fluid 204 can include any fluid capable of being cooled to the desired temperature and remaining a fluid. For example, the cooling fluid 204 can include glycol or another fluid. Glycol includes any organic compound with two hydroxyl (OH) groups attached to different carbon atoms. For example, glycol can include ethylene glycol (commonly called antifreeze). The cooling fluid 204 can then be cooled, allowing the bladder receptacle to be placed on the user's face, cooling the desired region. The cooling fluid 204 can be placed directly within the bladder receptacle 102 or can include separate packaging and be inserted into the bladder receptacle 102 as needed, as described below.

FIG. 2 further shows that the wire frame 108 can be located within the bladder receptacle 102. In at least one implementation, the wire frame 108 exerts a pressure on the bladder receptacle 102, conforming the bladder receptacle 102 to the desired shape. In particular, the bladder receptacle 102 can be flexible, allowing it to at least partially change its shape. The wire frame 108 can provide a rigidity, which allows the bladder receptacle 102 to retain the desired shape.

FIG. 2 also shows that he oral surgery recovery mask 100 can include micro suction elements 206. In at least one implementation, the micro suction elements 206 can help the oral surgery recovery mask 100 to remain in place. In particular, as the air is removed from in between the micro suction elements 206 and the user's skin, the external air pressure presses the oral surgery recovery mask 100 more firmly against the face of the user, helping the oral surgery recovery mask 100 to remain in place.

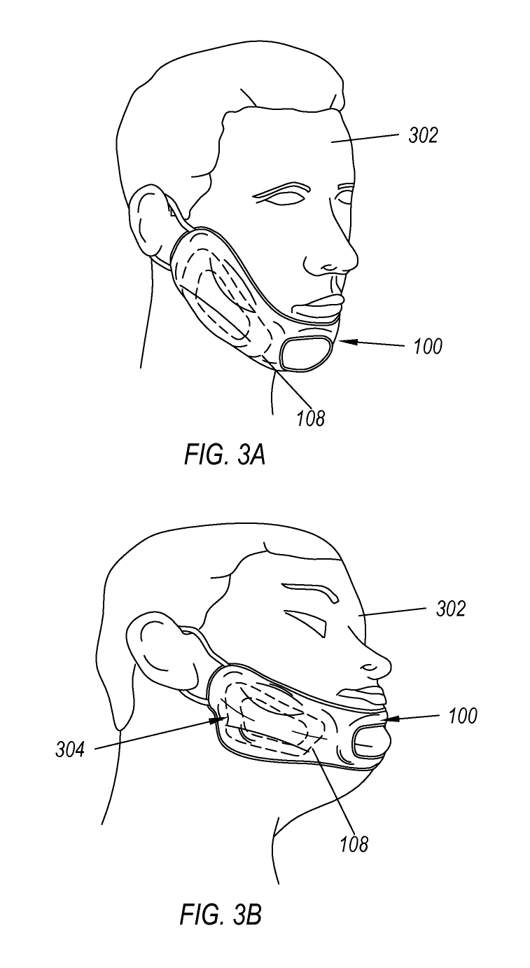

FIGS. 3A and 3B illustrate the oral surgery recovery mask 100 in use. FIG. 3A illustrates a perspective view of the oral surgery recovery mask 100 in use; and FIG. 3B illustrates a bottom perspective view of the oral surgery recovery mask 100 in use. In at least one implementation, the oral surgery recovery mask 100 can be placed on the face of a user 302. The oral surgery recovery mask 100 can be used to cool only the desired portion of the user's face. I.e., the oral surgery recovery mask 100 can target the cooling to increase the comfort of the user 302 and target the benefits of the cooling.

FIGS. 3A and 3B show that the oral surgery recovery mask adopts the shape of the face of the user 302. In particular, the wire frame 108 can bend 304. I.e., a moderate amount of force on the wire frame 108 can cause to change shape. The change in shape can allow the mask to more accurately conform to the face of the user 302. One of skill in the art will appreciate that the wire frame 108 can include more than one bend 304. I.e., multiple bends 304 can be created, changing the overall shape of the oral surgery recovery mask 100.

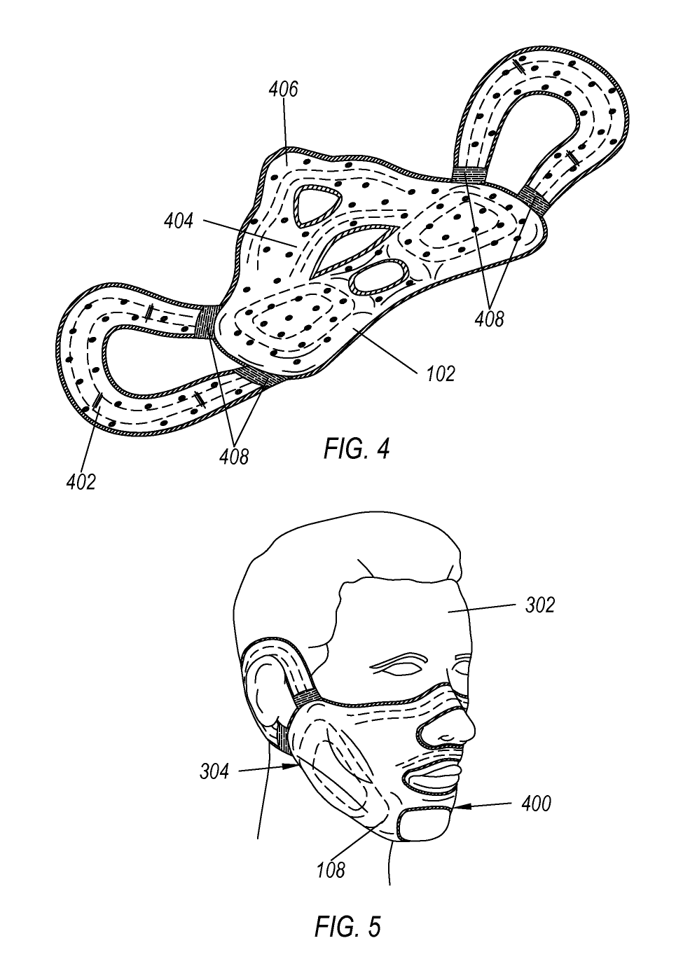

FIG. 4 illustrates an alternative example of an oral surgery recovery mask 400. In at least one implementation, the oral surgery recovery mask 400 can be used to cool alternative areas of the user's face. For example, the oral surgery recovery mask 400 can be used to cool the area around the user's mouth and nose. One of skill in the art will appreciate that an oral surgery recovery mask of any shape for cooling any desired area. Therefore, the oral surgery recovery mask 400 and the oral surgery recovery mask 100 of FIG. 1 are exemplary and not limiting.

FIG. 4 shows that the oral surgery recovery mask 400 can include a cooling ear strap 402. In at least one implementation, the cooling ear strap 402 can be used to secure the oral surgery recovery mask 400 in the desired location. In addition, the cooling ear strap 402 can be filled with cooling fluid. I.e., the ear strap 402 can include an additional cavity which is filled with cooling fluid, cooling the area around a user's ear.

FIG. 4 also shows that the oral surgery recovery mask 400 can include a mouth strap 404. In at least one implementation, the mouth strap 404 can cool the area around a user's mouth. I.e., the mouth strap 404 can be filled with cooling fluid. For example, the mouth strap 404 can include an additional cavity which is filled with cooling fluid, cooling the user's upper lip. Additionally or alternatively, the area between the mouth strap 404 can accommodate the user's lips, allowing access to the user's mouth.

FIG. 4 further shows that the oral surgery recovery mask 400 can include a nose strap 406. In at least one implementation, the nose strap 406 can cool the user's nose. I.e., the nose strap 406 can be filled with cooling fluid. For example, the nose strap 406 can include an additional cavity which is filled with cooling fluid, cooling the user's nose. Additionally or alternatively, the area between the nose strap 406 can accommodate the user's nose, allowing access to the user's nostrils.

FIG. 4 additionally shows that the oral surgery recovery mask 400 can include one or more attachments 408. In at least one implementation, the one or more attachments 408 can connect the ear straps 402, the mouth strap 404 and/or the nose strap 406 to the bladder receptacle 102. The one or more attachments 408 can be permanent or can be temporary. For example, the one or more attachments 408 can allow the ear straps 402, the mouth strap 404 and/or the nose strap 406 to be changed as desired. Additionally or alternatively, the one or more attachments 408 can include any desired material. For example, the one or more attachments 408 can include an elastic material, which allows the oral surgery recovery mask 400 to be adapted to different users. I.e., an elastic material in the one or more attachments 408 can allow the oral surgery recovery mask 400 to be placed on users with different sizes and shapes of faces.

FIG. 5 illustrates an example of the alternative oral surgery recovery mask 400 in use. FIG. 5. In at least one implementation, the oral surgery recovery mask 400 can be placed on the face of a user 302. The oral surgery recovery mask 400 can be used to cool only the desired portion of the user's face. I.e., the oral surgery recovery mask 400 can target the cooling to increase the comfort of the user 302 and target the benefits of the cooling.

FIGS. 5 show that the oral surgery recovery mask adopts the shape of the face of the user 302. In particular, the wire frame 108 can bend 304. I.e., a moderate amount of force on the wire frame 108 can cause to change shape. The change in shape can allow the mask to more accurately conform to the face of the user 302. One of skill in the art will appreciate that the wire frame 108 can include more than one bend 304. I.e., multiple bends 304 can be created, changing the overall shape of the oral surgery recovery mask 400.

FIG. 5 also shows that the one or more attachments 408 secure the ear straps 402, the mouth strap 404 and the nose strap 406 in the desired location. In particular, the one or more attachments 408 provide tension which secures the location of the ear straps 402, the mouth strap 404 and the nose strap 406. Additionally or alternatively, the ear straps 402, the mouth strap 404 and/or the nose strap 406 can include other mechanisms which help secure the ear straps 402, the mouth strap 404 and/or the nose strap 406. For example, the ear straps 402, the mouth strap 404 and/or the nose strap 406 can include microsuction elements, as described above.

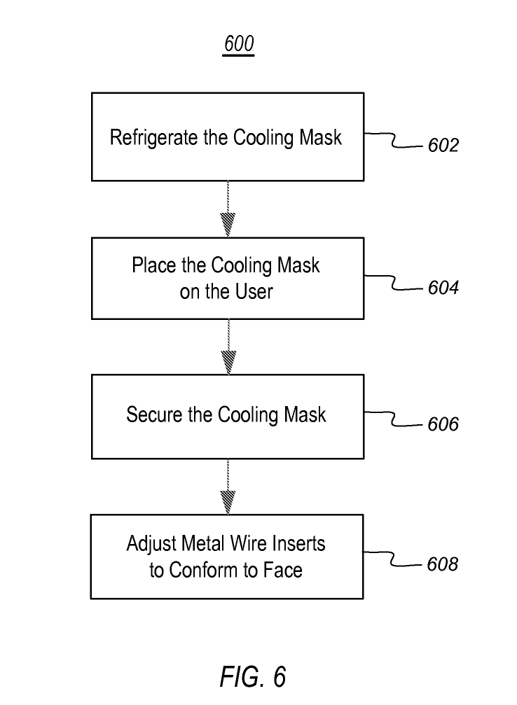

FIG. 6 is a flow chart illustrating a method 600 of using an oral surgery recovery mask 100. In at least one implementation, the oral surgery recovery mask can be the oral surgery recovery mask 100 of FIG. 1 or the oral surgery recovery mask 100 of FIG. 3. Therefore, the method 600 will be described, exemplarily, with reference to the oral surgery recovery mask 100 of FIG. 1 or the oral surgery recovery mask 100 of FIG. 3. Nevertheless, one of skill in the art can appreciate that the method 600 can be used with an oral surgery recovery mask other than the oral surgery recovery mask 100 of FIG. 1 or the oral surgery recovery mask 100 of FIG. 3.

FIG. 6 shows that the method 600 can include refrigerating 602 the oral surgery recovery mask. In at least one implementation, refrigerating 602 the oral surgery recovery mask can include reducing the temperature of the oral surgery recovery mask to any desired temperature. For example, the oral surgery recovery mask 600 can be placed in a fridge, freezer, cooler or any other device which lowers the temperature of the oral surgery recovery mask.

FIG. 6 also shows that the method 600 can include placing 604 the oral surgery recovery mask on the user. In at least one implementation, the oral surgery recovery mask can be placed with the bladder receptacle near the area to be cooled. The oral surgery recovery mask can then remove heat from the desired area. I.e., the oral surgery recovery mask can target an area on the user to cool.

FIG. 6 further shows that the method 600 can include securing 606 the oral surgery recovery mask. In at least one implementation, the oral surgery recovery mask can be secured 606 in any desired method. For example, the oral surgery recovery mask can be secured using one or more ear straps. Additionally or alternatively, the oral surgery recovery mask can include one or more micro suction elements for retaining the mask in the desired area. Additionally or alternatively, one or more wires within the oral surgery recovery mask can be molded to the user.

FIG. 6 additionally shows that the method 600 can include adjusting 608 the metal wire inserts to conform to the user's face. In at least one implementation, adjusting 608 the metal wires can ensure that the oral surgery recovery mask maintains contact along the user's face. I.e., the metal wires can be adjusted 608 to allow the mask to be customized to each user. That is, different users can use the same or similar masks which are adjusted 608 to the shape of their face.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An oral surgery recovery mask for cooling an area of a user's face, the oral surgery recovery mask comprising:
a bladder receptacle, wherein the bladder receptacle includes:
a first layer; and
a second layer at least partially secured to the first layer;
a cooling liquid, wherein the cooling fluid is configured to be inserted into the bladder receptacle; and
a securing device, wherein the securing device is configured to hold the oral surgery recovery mask in place relative to the user.

2. The oral surgery recovery mask of claim 1, wherein the cooling fluid includes glycol.

3. The oral surgery recovery mask of claim 1, wherein the cooling fluid includes a cold pack.

4. The oral surgery recovery mask of claim 1, wherein the bladder receptacle includes an opening, wherein the opening is configured to allow the user to add or remove cooling fluid.

5. The oral surgery recovery mask of claim 1, wherein the securing device includes at least one ear strap.

6. The oral surgery recovery mask of claim 5, wherein the securing device includes a second ear strap.

7. The oral surgery recovery mask of claim 5, wherein the ear strap includes an elastic material.

8. The oral surgery recovery mask of claim 1, wherein the securing device includes a micro suction element.

9. The oral surgery recovery mask of claim 8, wherein the micro suction element is configured to adhere the oral surgery recovery mask to the user's skin.

10. The oral surgery recovery mask of claim 1 further comprising a wire frame, wherein the wire frame is configured to conform the shape of the oral surgery recovery mask to the shape of the user's face.

11. The oral surgery recovery mask of claim 10, wherein the wire frame is located inside the bladder receptacle.

12. An oral surgery recovery mask for cooling an area of a user's face, the oral surgery recovery mask comprising:
a bladder receptacle, wherein the bladder receptacle includes:
a first layer, wherein the first layer includes plastic; and
a second layer, wherein the second layer:
includes plastic; and
at least partially secured to the first layer;
a cooling liquid, wherein the cooling fluid is configured to be inserted into the bladder receptacle;
a securing device, wherein the securing device includes:
a first ear strap;
a second ear strap; and
micro suction elements, wherein the micro suction elements are configured to adhere the bladder to the face of the user; and
a wire frame, wherein the wire frame is configured to allow the user to change at least a portion of the shape of the oral surgery recovery mask.

13. The oral surgery recovery mask of claim 12, wherein the bladder receptacle includes a chin hole.

14. The oral surgery recovery mask of claim 12 further comprising a nose strap.

15. The oral surgery recovery mask of claim 12, wherein the wire frame is located within the bladder receptacle.

16. The oral surgery recovery mask of claim 12 further comprising a mouth strap.

17. The oral surgery recovery mask of claim 12, wherein the first ear strap includes:
a cavity, wherein the cavity includes the cooling fluid.

18. The oral surgery recovery mask of claim 17, wherein the first ear strap includes:
an attachment, wherein the attachment:
is configured to attach the ear strap to the bladder receptacle; and
includes an elastic material.

19. A method for using an oral surgery recovery mask for cooling an area of a user's face, the method comprising:
refrigerating the oral surgery recovery mask;
placing the oral surgery recovery mask on a user; and
securing the oral surgery recovery mask on the user.

20. The method of claim 19, wherein refrigerating the oral surgery recovery mask includes reducing the temperature of the oral surgery recovery mask.
